(19) United States
(12) Patent Application Publication
(10) Pub. No.: US 2026/0216434 A1
(43) Pub. Date: Jul. 30, 2026

(54) A SUBASSEMBLY OF A MEDICAMENT DELIVERY DEVICE

(71) Applicant: SHL Medical AG, Zug (CH)

(72) Inventors: Amit Singh, Deerfield Beach, FL (US); Iskender Okan Ates, Deerfield Beach, FL (US)

(21) Appl. No.: 19/146,389

(22) PCT Filed: Dec. 13, 2023
(86) PCT No.: PCT/EP2023/085563

§ 371 Date: Jul. 8, 2025

Related U.S. Application Data
(60) Provisional application No. 63/439,344, filed on Jan. 17, 2023.

(30) Foreign Application Priority Data
Mar. 6, 2023 (EP) 23160094.1

(51) Int. Cl. A61M 5/20; A61M 5/32

(52) U.S. Cl. CPC A61M 5/2033; A61M 5/3245; A61M 5/326; A61M 2005/2013; A61M 2005/206; A61M 2005/208; A61M 2005/3247; A61M 2005/3267; A61M 2202/0007; A61M 2207/00

(57) ABSTRACT

The present disclosure provides a subassembly of a medicament delivery device. The subassembly includes a cover structure with a first latching element and a retaining structure having an envelope surface arranged radially outside of the cover structure and having a second latching element. In a first extended position of the cover structure, the first latching element is out of engagement and positioned distally with respect to the second latching element. In a second extended position, the first latching element is in mutual engagement with respect to the second latching element, such that the cover structure is prevented from moving towards a retracted position. In the second extended position, the envelope surface covers or surrounds the first and/or the second latching element, in order to prevent disengagement of the first latching element and the second latching element by a user.

### TECHNICAL FIELD

The present invention generally relates to medicament delivery devices, such as autoinjectors, and particularly concerns a subassembly of such a medicament delivery device, which is adapted to prevent injuries to a user caused by a medicament delivery element at a proximal end of the medicament delivery device.

### BACKGROUND

A number of medical conditions require injections. These days, a number of different injection devices (also called medicament delivery devices) exist, including various types of pen injectors, autoinjectors and on-body devices. Although many of these devices have enabled major improvements in the management of a number of medical conditions, various limitations do still exist in the current technology. A particular challenge relates to the development of medicament delivery devices that are not only easy to handle for the user, but that are also particularly safe. Especially patients who require frequent injections and patients who need to inject particularly viscous medicaments face the problem that sooner or later a malfunction or an incorrect handling of the device might result in an injury. Also patients, who are not experienced in handling the medicament delivery device, may operate it incorrectly, what might lead to injuries. In this context, the medicament delivery element in particular, e.g. the needle through with the medicament is injected subcutaneously, poses a risk of patient injury.

In order to address these challenges, devices are known, which comprise a cover structure that surrounds the medicament delivery element, in order to prevent the user from getting into contact with it before and after the injection. Only for the actual injection, the cover structure is retracted into the housing of the device, such that the medicament delivery element, e.g. the needle, is exposed.

Document WO 2021/110344 A1 discloses an injector with a cover structure that surrounds the delivery needle. The cover structure is spring-loaded towards the proximal direction, i.e. in the direction of the needle's tip. By simply pushing the cover structure distally, however, it still possible for the user to expose the needle before and after the injection.

In the yet unpublished patent application EP 21 212 721.1 of the same applicant, an autoinjector with a retractable cover structure is disclosed. When the cover structure is moved from its extended position in a retracted position for medicament injection, a guide protrusion of the cover structure engages a guiding track of a rotator, which results in a slight rotation of the cover structure. When the cover structure is subsequently, i.e. after completed injection, moved back into the extended position, the guide protrusion engages a locking portion of the rotator and at the same time a radially outwardly directed locking protrusion of the cover structure is moved radially outwards, such that it engages a flexible portion of the housing, which permanently locks the cover structure in its extended position. The design with the rotator and the guiding track is relatively complicated and requires adherence to tight manufacturing tolerances. Moreover, it cannot be adequately guaranteed that a user cannot forcibly move the cover structure from its extended position.

Documents US 2020/0384208 A1, U.S. Pat. No. 10,646,654 B2 and U.S. Pat. No. 10,398,848 B2 disclose injector devices that comprise a cover structure, which serves to surround a medicament delivery element and has radially extending lockout snaps, in order to lock the cover structure in an extended position after completed injection. Before and during injection, the lockout snaps are arranged at the inside of a surrounding housing. After the injection, the cover structure is moved into an extended position, with the lockout snaps coming to lie outside of the housing. As a result, the lockout snaps flex out and engage with the proximal rim of the housing, which prevents the cover structure to be pushed towards its retracted position. The lockout snaps, however, can still be pinched inwardly by a user to unlock the device. Furthermore, the lockout snaps are in a stressed state during transport and storage of the device, i.e. from manufacture until application by the user, which can lead to material creep and lockout failure.

### SUMMARY

It is an object of the present invention to provide a subassembly of a medicament delivery device, which is not only easy to manufacture and handle, but also particularly safe to use.

This object is solved by a subassembly as claimed in claim 1. Further embodiments of the subassembly are provided in the dependent claims. A medicament delivery device comprising such a subassembly is indicated in claim 13.

The present invention thus provides a subassembly of a medicament delivery device for delivering a medicament from a medicament container to a human or animal patient, the medicament delivery device having a medicament delivery element with a proximal end, through which the medicament can be delivered to the patient, wherein the subassembly comprises:
a cover structure configured to surround the medicament delivery element and having a first latching element, the cover structure being movable along an axial direction from a proximal first extended position to a distal retracted position and to a proximal second extended position, in order to cover the medicament delivery element in the first extended position before medicament delivery and in the second extended position after medicament delivery and to expose the medicament delivery element in the retracted position during medicament delivery; and
a retaining structure having an envelope surface arranged radially outside of the cover structure and having a second latching element.

In the first extended position, the first latching element is out of engagement with respect to the second latching element, and arranged (positioned) distally with respect to the second latching element.

In the second extended position, the first latching element is in mutual engagement with respect to the second latching element, such that the cover structure is prevented from moving towards the retracted position.

In the second extended position, the envelope surface covers or surrounds the first latching element and/or the second latching element, in order to prevent disengagement of the first latching element and the second latching element by a user.

Due to the covering or surrounding of the first latching element and/or the second latching element by means of the envelope surface in the second extended position, an intentional or unintentional disengagement of the first and the second latching element by the user can effectively be prevented. This is achieved by covering the site, where the two latching elements are engaged, by means of the envelope surface of the retaining structure or by surrounding one or both latching elements by means of the envelope surface, in such a way that a user does not have access to the site of engagement of the two latching elements. Since the user does not have any access to the two mutually engaged latching elements, when cover structure is in its second extended position, the risk of a malfunction or an incorrect handling of the medicament delivery device by the user is effectively reduced. The device can also be manufactured easily and with a relatively small number of parts, because the envelope surface, which forms the covering or surrounding element of the engagement site, is part of the retaining structure which also comprises the second latching element.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose or medicament delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

When a component is said to move proximally, distally, axially in a proximal direction, axially in a distal direction or equivalent terms, the movement is relative to the retaining structure (housing) of the medicament delivery device, unless mentioned otherwise.

If the envelope surface covers the first latching element and/or the second latching element in the second extended position of the cover structure, the envelope surface usually covers the first latching element and/or the second latching element radially towards the outside. Thus, in this case, the envelope surface extends radially over the two latching elements. Due to this covering by means of the envelope surface, the user preferably does not have any access from a radial direction to the site, where the two latching elements are engaged, advantageously not with his fingers and more advantageously also not with a tool.

If the envelope surface surrounds the first latching element and/or the second latching element in the second extended position of the cover structure, the envelope surface usually covers the first latching element and/or the second latching element along the circumferential direction, preferably along both mutually opposite circumferential directions. Thus, the word “surrounding” is to be understood with regard to the first latching element and/or the second latching element here, i.e. it is the first latching element and/or the second latching element, which is laterally surrounded by the envelope surface. Thus, in this case, the envelope surface covers the two latching elements along at least one circumferential direction, preferably along both circumferential directions. In a particularly preferred embodiment, the envelope surface covers the two latching elements not only along one or both circumferential directions, but also along at least one, preferably along both axial directions, i.e. along both the proximal and the distal directions. Particularly preferred is an embodiment in which the two latching elements are completely surrounded by the envelope surface in the second extended position of the cover structure. The second latching element of the retaining structure can particularly be arranged within a through-going aperture, i.e. a hole, or within a non-through going aperture, i.e. a local depression, of the envelope surface. Due to this partial or complete surrounding of the latching elements by means of the envelope surface, the user preferably does not have any access from a circumferential and/or axial direction to the site, where the two latching elements are engaged, advantageously not with his fingers and more advantageously also not with a tool.

The medicament delivery device can for example be an autoinjector, a pen injector or an on-body device. The medicament delivery element is usually a cannula or hollow needle, also abbreviated as a needle. The medicament delivery device is preferably adapted to the be inserted into the skin of the patient with its proximal end first, in order to subcutaneously inject the medicament to the desired place inside of the patient's body. The medicament is usually delivered from the medicament container to the patient, by means of forwarding a plunger inside of the container along the proximal direction. The forwarding of the plunger can be performed manually, motor-driven, or, what is preferred here, by means of a spring, in particular a spiral spring.

The cover structure preferably comprises a sleeve at its proximal end, which completely surrounds the medicament delivery element in the second extended position of the cover structure, in order to prevent access to the medicament delivery element from all radial directions, preferably before and after the injection. Thus, in the first and second extended position, the medicament delivery element is preferably covered by the cover structure, in particular by the sleeve of the cover structure, in all radial directions. In its retracted position, the cover structure is preferably axially displaced along the distal direction with respect to the retaining structure as compared to the first and second extended position. The first extended position of the cover structure is preferably arranged between the retracted position and the second extended position, with the first extended position preferably being by a multiple closer to the second extended position than to the retracted position.

In the context of the present document, “prevent disengagement of the first latching element and the second latching element by a user” is not to be understood that a disengagement of the latching elements is not possible for a user by all means and under all circumstances. Instead, it is to be understood that due to the respective feature, it is made considerably more difficult for a user to disengage the latching elements. In particular, it is made more difficult for a user to disengage the latching elements by means of his own hands, without using any tools. In a preferred embodiment, a disengagement of the latching elements is made more difficult for a user even when applying a tool.

The retaining structure can generally be formed any component of the medicament delivery device. Preferred, however, is an embodiment, in which the housing, in particular the outer housing, of the medicament delivery device forms the retaining structure. The retaining structure, in particular if it is formed by the housing, preferably has a cylindrical wall, which forms the envelope surface. In this case, the cover structure is preferably radially arranged within this cylindrical wall. Embodiments are conceivable, however, where the envelope surface does not circumferentially extend around the cover structure, but only over e.g. a certain angular range. The envelope surface, however, has a certain extension along the circumferential direction as well as a certain extension along the axial direction, which allows the envelope surface to radially cover or laterally surround the first latching element and/or the second latching element. If the retaining structure is not formed by the outer housing, it can for example be formed by a sleeve element arranged within the housing.

In the first extended position, which is taken before medicament delivery, the cover structure can preferably be moved along the distal direction relative to the retaining structure, because the first latching element of the cover structure is out of engagement with respect to the second latching element and placed distally relative to the second latching element. In the retracted position, i.e. during medicament delivery, the first latching element is also distal and out of engagement with respect to the second latching element. In the second extended position, which is taken after medicament delivery, such a movement of the cover structure along the distal direction relative to the retaining structure is not possible anymore, because the first latching element is then arranged proximal and in engagement with respect to the second latching element.

The first latching element or the second latching element is preferably a snap-in element. A snap-in element is an element that preferably has some flexibility to automatically snap into the complementary other latching element when engaged therewith due to a bias. The snap-in element can particularly be in the form of a hook, a flexible arm, or an arm with a hook. The snap-in element is thus preferably formed by that latching element which moves more strongly during snap-in. The latching element, which is not the snap-in element, preferably comprises a stop surface which comes into engagement with the snap-in element in the second extended position of the cover structure in such a way, that a further movement of the cover structure along the distal direction relative to the retaining structure is effectively prevented. In certain embodiments, one of the latching elements can be formed exclusively by a stop surface. The stop surface can for example be a rim of the housing or of the cover structure. If the snap-in element is formed by the first latching element, the stop surface formed by the second latching element preferably faces in the proximal direction. If the snap-in element is formed by the second latching element, the stop surface formed by the first latching element preferably faces in the distal direction.

If the first latching element of the cover structure is the snap-in element, then the snapping-in of the first latching element, when engaging with the second latching element, preferably occurs along a radial outward direction. If the second latching element of the retaining structure is the snap-in element, then the snapping-in of the second latching element, when engaging with the first latching element, preferably occurs along a radial inward direction.

In a particularly preferred and easy-to-manufacture embodiment, the first latching element is the snap-in element and the second latching element is formed by a depression on an inner face of the envelope surface of the retaining structure. Thus, in this embodiment, the envelope surface usually covers the first latching element and the second latching element radially towards the outside in the second extended position of the cover structure. Due to this covering by the envelope surface of the retaining structure, which is preferably formed by the outer housing of the device, both latching elements are preferably at the inside of the retaining structure in the second extended position of the cover structure, such that an access to the latching elements is not possible for the user. In this embodiment, the snap-in element is preferably formed by a radially outwardly oriented flexible hook. The flexible hook can particularly be formed by a free end of an axially extending guide arm of the cover structure.

In the embodiment as mentioned, an axial guide groove can be provided on the inner face of the envelope surface, in order to guide the snap-in element in the movement of the cover structure from the first extended position to the retracted position. The guide groove is also advantageous as it allows the snap-in element to be in a relaxed state both in the first extended position and in the retracted position. Since the cover structure usually is in the first extended position during transport and storage of the device, having the snap-in element in a relaxed state is particularly advantageous. Material creep and/or material fatigue can be avoided in this way.

The snap-in element preferably has a chamfered surface on its side facing in the proximal direction and/or the axial guide groove is axially terminated in the proximal direction by a chamfered surface. The provision of a chamfered surface on the snap-in element and/or on the guide groove facilitates the movement of the cover structure from the retracted position to the second extended position.

In another, also particularly preferred and easy-to-manufacture embodiment, the second latching element is the snap-in element, which can particularly be formed by a flexible arm. The first latching element can then be in the form of e.g. a stop surface, which can for example be formed by a distal rim or by a through-going or non-through-going aperture (i.e. a local depression on the inner face) of the cover structure. Thus, in this embodiment, in the second extended position of the cover structure, the envelope surface usually surrounds the second latching element along the circumferential and/or axial directions. Due to this lateral surrounding of the second latching element, it is not possible for the user to disengage the first latching element and the second latching element in the second extended position of the cover structure. It might be possible for the user to press the second latching element radially inwards, but not outwards. When pressing the second latching element radially inwards, the latching element is preferably only pressed against a further element of the medicament delivery device which is arranged further inside of the device. As a result, the mutual engagement of the two latching elements cannot be released. Pulling the second latching element radially outwards is preferably effectively prevented in this embodiment due to the surrounding with the envelope surface, which does not allow a respective access to the second latching element. The second latching element can particularly be arranged within a through-going aperture (i.e. within a hole) or within a non-through-going aperture (i.e. within a local depression) of the envelope surface. In this embodiment, the snap-in element is preferably formed by a radially inwardly oriented flexible arm or hook. In an unstressed state, the snap-in element preferably extends radially inwards from the envelope surface. Thus, the retaining structure is advantageously moulded such that the snap-in element extends radially inwards.

Generally preferred are embodiments, in which the cover structure has two or more first latching elements and the retaining structure has two or more second latching elements, such that two or more pairs of a first latching element and a second latching element are provided. The pairs of a first latching element and a second latching element are preferably distributed, in particular regularly distributed, along a circumferential direction of the subassembly. By having more than one pair of latching elements, it can be better achieved that the cover structure remains central in the device, particularly its second extended position. As a result, reliable prevention from moving towards the retracted position can be achieved even with large manufacturing tolerances.

Manufacturing of the subassembly becomes particularly easy, if the cover structure and/or the retaining structure are each made as a whole in one piece. Particularly preferred is an embodiment, in which the cover structure and/or the retaining structure are each made, e.g. from a plastic material, by means of injection moulding.

The present invention further provides a medicament delivery device comprising the subassembly as indicated.

Exemplary types of drugs or medicaments that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 schematically shows a perspective view of a medicament delivery device in the form of an autoinjector comprising a subassembly according to a first inventive embodiment, with extended cover structure;

FIG. 2 shows the medicament delivery device of FIG. 1, with retracted cover structure;

FIG. 3 shows the medicament delivery device of FIG. 1, with extended cover structure and removed housing;

FIG. 4 shows the medicament delivery device of FIG. 3, with additionally removed cover structure;

FIG. 5 shows the medicament delivery device of FIG. 4, with additionally removed syringe container and needle;

FIG. 6 shows the medicament delivery device of FIG. 5, with additionally removed piston and rotator;

FIG. 7 shows a perspective partial view of the distal end of the cover structure of the medicament delivery device of FIG. 1;

FIG. 8 shows a perspective partial view of the distal end of the housing of the medicament delivery device of FIG. 1;

FIG. 9 shows a first central cross-sectional view through the housing of the medicament delivery device of FIG. 1;

FIG. 10 shows a second central cross-sectional view through the housing of the medicament delivery device of FIG. 1, from a direction perpendicular to the viewing direction of FIG. 9;

FIG. 11 shows a central cross-sectional view through the distal part of the medicament delivery device of FIG. 1, prior to medicament delivery and with the cover structure in its first extended position;

FIG. 12 shows a central cross-sectional view through the distal part of the medicament delivery device of FIG. 1, immediately prior to medicament delivery and with the cover structure in its retracted position;

FIG. 13 shows a central cross-sectional view through the distal part of the medicament delivery device of FIG. 1, immediately after medicament delivery and with the cover structure in its retracted position;

FIG. 14 shows a central cross-sectional view through the distal part of the medicament delivery device of FIG. 1, after medicament delivery and with the cover structure in its second extended position;

FIG. 15 schematically shows a perspective view of a medicament delivery device in the form of an autoinjector comprising a subassembly according to a second inventive embodiment;

FIG. 16 shows the medicament delivery device of FIG. 15, with removed front cap and housing;

FIG. 17 shows the medicament delivery device of FIG. 16, with additionally removed cover structure;

FIG. 18 shows the medicament delivery device of FIG. 17, with additionally removed cover spring, needle shield and rear cap;

FIG. 19 shows a central cross-sectional view through the distal part of the medicament delivery device of FIG. 15, prior to medicament delivery and with the cover structure in its first extended position;

FIG. 20 shows a central cross-sectional view through the distal part of the medicament delivery device of FIG. 15, immediately after medicament delivery and with the cover structure in its retracted position; and

FIG. 21 shows a central cross-sectional view through the distal part of the medicament delivery device of FIG. 15, after medicament delivery and with the cover structure in its second extended position.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Elements, components and parts of different examples, embodiments or variants, but having the same or a similar function are designated by the same reference numerals in the figures.

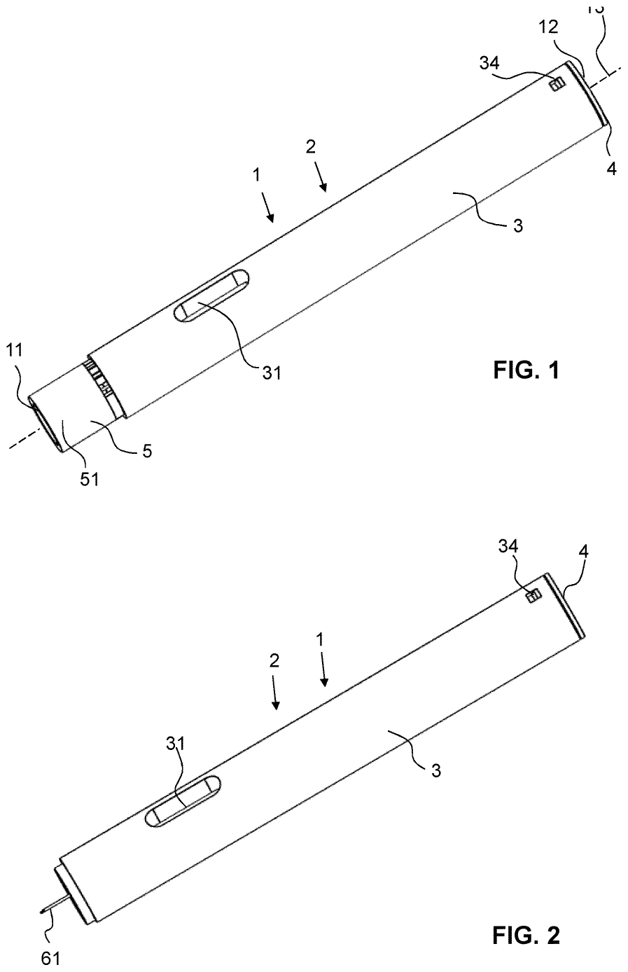

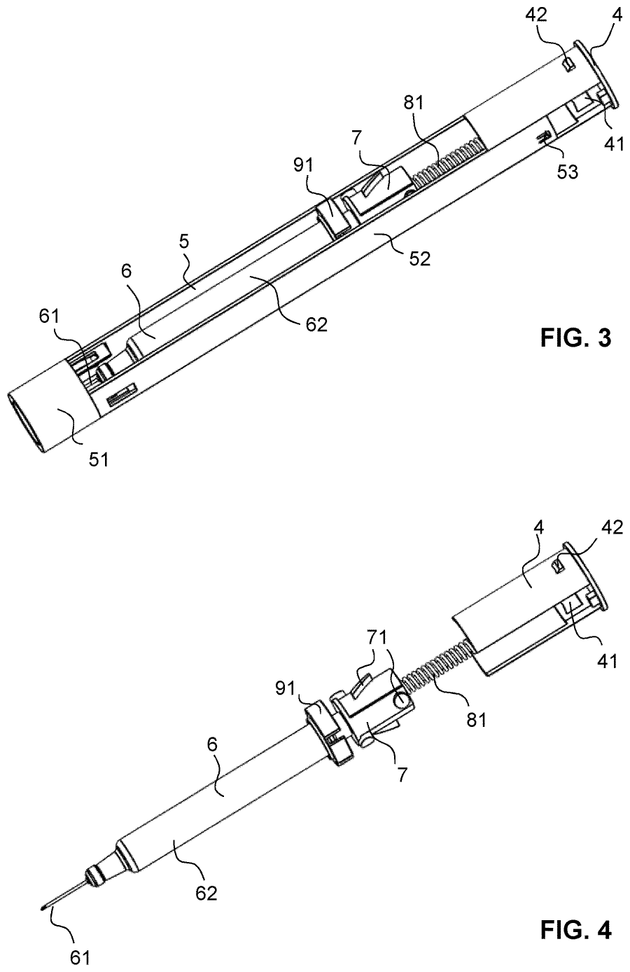

FIGS. 1 and 2 show an example of a medicament delivery device 1 such as an autoinjector according to an inventive embodiment. The medicament delivery device 1 is configured to expel medicament from a medicament container 62 (see FIG. 3) via a medicament delivery element 61 to a user at a dose delivery site. The medicament container 62 may be a syringe. The medicament delivery device 1 extends from a proximal end 11 to a distal end 12 relative to a longitudinal main axis 13.

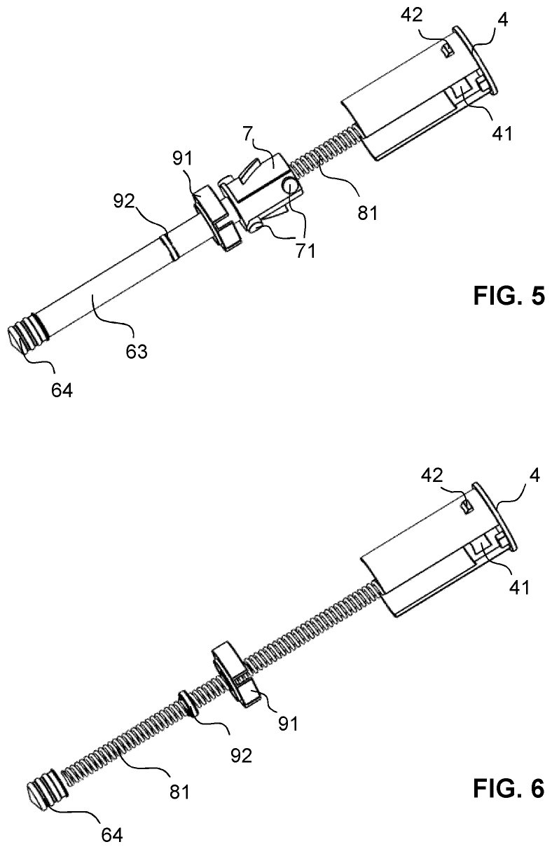

The medicament delivery device 1 comprises a housing 3 with a window 31, which allows the user to see e.g. the filling level of the container 62 and/or the position of a plunger 64 (FIG. 5) used for expelling the content from the container 62. A rear cap 4 covers the distal end of the housing 3. The housing 3 generally has a hollow cylindrical design, by which the longitudinal main axis 13 is defined.

The medicament delivery device 1 comprises a cover structure 5. The cover structure is commonly called needle cover. The cover structure 5 is arranged within the housing 3 and extends proximally from the proximal end of the housing 3. The housing 3, which forms a retaining structure with regard to the cover structure 5 as will be explained further below, and the cover structure 5 together form a subassembly 2.

The cover structure 5 is configured to be moved linearly relative to the housing 3 along the longitudinal main axis 13 from a first extended position shown in FIG. 1 to a retracted position shown in FIG. 2, in which the cover structure 5 is received further in the housing 3 and in which a medicament delivery element 61 such as a needle is exposed at the proximal end 11 of the medicament delivery device 1. After completion of the injection, the cover structure 5 is moved back in proximal direction and into a second extended position. When the medicament delivery device 1 is viewed from the outside, the second extended position of the cover structure 5 is similar to the first extended position as shown in FIG. 1.

The cover structure 5 comprises a proximal portion with a sleeve 51, which serves to surround the medicament delivery element 61 in the first and the second extended position of the cover structure 5, as it is shown in FIG. 1. Thus, in both extended positions, the user cannot be injured by the medicament delivery element 61 due to the cover structure 5. The cover structure 5 is biased in the proximal direction towards the extended position shown in FIG. 1.

The first extended position is thus adopted by the cover structure 5 before medicament injection. The retracted position as shown in FIG. 2 is only adopted during injection. After injection, the cover structure 5 adopts its second extended position. In the second extended position, the cover structure 5 proximally protrudes slightly further from the housing 3 than in the first extended position.

For the injection, the user places the proximal end 11 of the medicament delivery device 1 on the desired site on his own or another patient's body and presses the device in the proximal direction. Due to the pressing action, the cover structure 5 is moved distally into the housing 3 until the retracted position is as shown in FIG. 2 is reached. At the same time, the patient's skin is pierced by the medicament delivery element 61 and the proximal portion of the medicament delivery element 61 is introduced in the patient's body. Upon reaching the retracted position, the cover structure 5 causes the medicament delivery device 1 to be activated and the injection to take place. The injection is effected by releasing an injection spring 81, as it is shown in FIG. 3, inside of the housing 3. Release of the injection spring causes the plunger 64 to be moved in the proximal direction, such that the content of the medicament container 62 is expelled through the medicament delivery element 61 into the patient's body.

After completion of the medicament delivery, the medicament delivery device 1 is withdrawn from the patient's body by the user. In doing so, the medicament delivery element 61 is removed from the patient's body and the cover structure 5, due to its bias, is moved into the second extended position. The second extended position adopted by the cover structure 5 after medicament delivery is similar, but differs somewhat from the one adopted first extended position before medicament delivery as will be explained further below.

FIGS. 3 to 6 show the inner parts of the medicament delivery device 1. Arranged in the proximal portion of the medicament delivery device 1 is a syringe 6 comprising the medicament container 62, in which the medicament to be injected is contained, and the medicament delivery element 61.

For expelling the medicament from the container 62 through the medicament delivery element 61, a piston 63 and a plunger 64 are arranged at least partially inside of the container 62. The plunger 64 is arranged at the proximal end of the piston 63 in such a way, that it can be pushed proximally by the piston 63 for medicament injection. The proximal pushing of the piston 63 and the plunger 64 is effected by the injection spring 81, which distally abuts the rear cap 4 and proximally the piston 63 and/or the plunger 64. In order to center and axially guide the injection spring 81, a guidance element can be provided at the rear cap 4, which axially extends from the rear cap 4 into the injection spring 81 along the proximal direction (not visible in the figures). Axial guidance of the piston 63 is provided by means of a first collar 92 with respect to the container 62 and by means of a second collar 91 with respect to the housing 3.

The rear cap 4 comprises two arms, which each extend axially into the housing 3 and each comprise a radially outwardly directed catch element 42. The catch elements 42 serve to engage with an aperture 34 provided in the distal end region of the housing 3 (see FIGS. 1 and 2), in order to secure the rear cap 4 to the housing 3 in the assembled state of the medicament delivery device 1.

The cover structure 5 comprises two guide arms 52, which are arranged at diametrically opposed positions at the distal end of the sleeve 51. From the sleeve 51, the guide arms 52 extend distally along the axial direction through a large part of the housing 3. Thus, the cover structure 5 is arranged radially inside of the housing 3. At the distal end of the housing 3, the guide arms 52 come to lie between the arms of the rear cap 4, such that the cover structure 5 is axially movable, but rotationally fixed with respect to the rear cap 4 and the housing 3.

At the distal end of the piston 63, a rotator 7 is arranged. The rotator 7 is axially and rotationally movable within the housing 3 and serves to release the injection spring 81 for medicament injection. For this purpose, the rotator 7 comprises radially outwardly directed guide protrusions 71, some of which engage a slotted guide system 41 provided on the radial inner surface of the rear cap 4. Before medicament delivery, which the cover structure 7 is still in its first extended position, the slotted guide system 41 prevents a displacement of the rotator 7 along the proximal direction. By pushing the cover structure 7 towards its retracted position, some of the guide protrusions 71 are engaged with radially inwardly directed driving elements that are provided on the inner surfaces of the guide arms 52 of the cover structure 5 (not visible in the figures). Due to this engagement of some of the guide protrusions 71 with the driving elements of the cover structure 5, the rotator 7 is rotated about the longitudinal main axis 13 upon further distal movement of the cover structure 5. Because of this rotation, the guide protrusions 71 are disengaged of the slotted guide system 41, what allows the rotator 5 to be displaced along the proximal direction. This releases the previously blocked injection spring 81 to move the rotator 5 and, thus, the piston 63 and the plunger 64 in the proximal direction. Thus, the medicament injection is performed. The person skilled in the art knows how to design and arrange the guide projections 71, the slot guide system 41 and the drive elements in such a way that the functionality described above is achieved.

Cross-sectional views of the housing 3, which is manufactured as a whole in one piece, are shown in FIGS. 9 and 10. As can be seen, the housing 36 comprises a guide sleeve 36 which is arranged at the inside of the main cylindrical wall of the housing 3 and serves to center the medicament container 62. The window 31 extends transversally through the housing 3 and in particular through the guide sleeve 36.

The “lockout-functionality” of the cover structure 5 is now explained based on FIGS. 11 to 14:

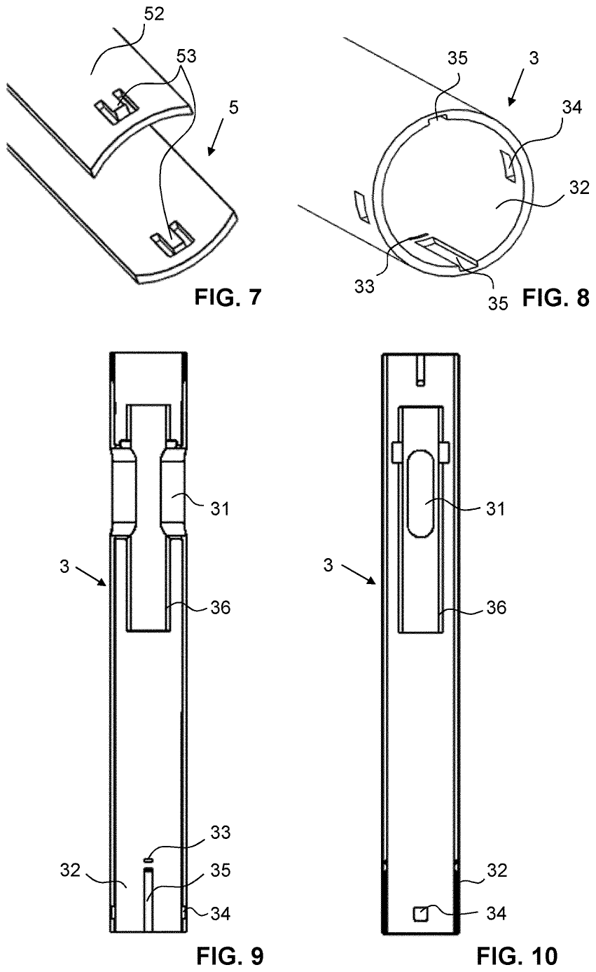

For locking the cover structure 5 in its second extended position after completion of the medicament injection, a latching element 53 is provided in the distal region of each of the guide arms 52. In the present embodiment, the latching element 53, which is best shown in FIG. 7, is provided within a rectangular aperture of the guide arm 52 and is formed by means of a flexible arm that extends in the distal direction and comprises a radially outwardly directed hook at its free end. The latching element 53 forms a snap-in element that is configured to engage a latching element 33 in the second extended position of the cover structure 5 and an axial guide groove 35 in the both the first extended position and the retracted position of the cover structure 5.

The axial guide groove 35 is arranged on the inner surface of the distal part of the housing 3 and extends linearly along the main longitudinal axis 13, as it is shown in FIG. 8. The latching element 33 is formed by a local, non-through going aperture, i.e. by a local depression, in the inner surface of the housing 3 arranged directly proximally to the axial guide groove 35.

In the first extended position, which is adopted prior to medicament injection, the cover structure 5 is arranged such within the housing 3, that the hook formed by each latching element 53 of the cover structure 5, engages one of the axial guide grooves 35 of the housing 3 at the most proximal position of the axial guide groove 35, as it is shown in FIG. 11. Due to the engagement with the axial guide grooves 35, the latching elements 53 are in an unstressed state in the first extended position.

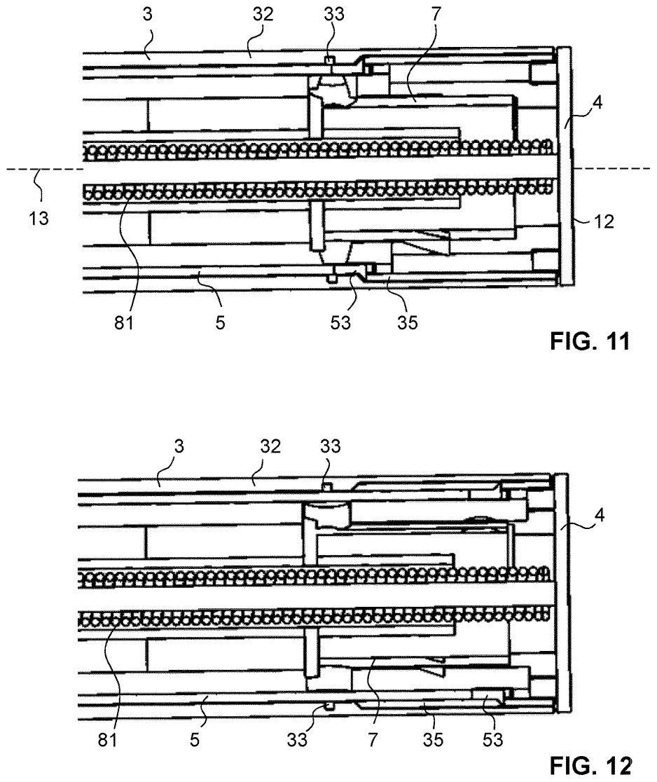

When the cover structure 5 is retracted immediately before medicament delivery, the latching structures 53 of the cover structure 5 are moved distally along their respective axial guide groove 35 until the cover structure reaches its retracted position as shown in FIG. 12.

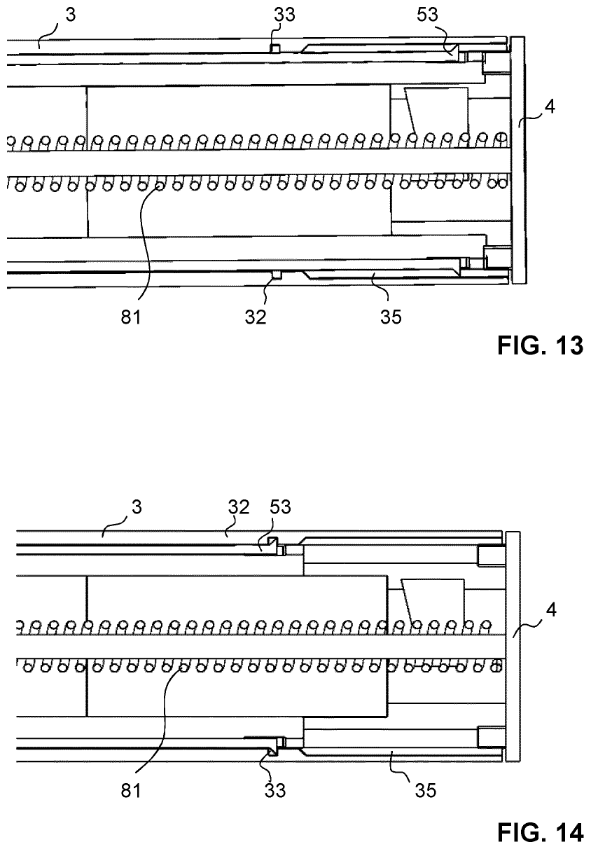

Due to the distal movement of the cover structure 5 relative to the housing 3, the rotator 7 is rotated and thereby releases the injection spring 81 to expel the medicament through the medicament delivery element 61. The respective state of the medicament delivery device 1 immediately after completed injection is shown in FIG. 13.

Due to the withdrawal of the medicament delivery device 1 after the injection by the user, the cover structure 5 is moved proximally until the latching elements 53 get into contact with the proximal ends of their respective axial guide groove 35. Since the hook formed by the latching element 53 has a chamfered surface on its side facing in the proximal direction and the axial guide groove 35 is axially terminated in the proximal direction by a chamfered surface in each case, the latching elements 53 are slightly bent radially inwards upon further proximal movement of the cover structure 5, such that the hook formed by each latching element 53 is allowed to be axially moved beyond the proximal end of the respective axial guide groove 35. Upon further axial movement, the hook of each latching element 53 engages with a latching element 33 of the housing 3, by snapping-in in the respective aperture in the radial direction outwards, as it is shown in FIG. 14. A further proximal movement of the cover structure 5 is prevented by a stop surface of e.g. the housing 3, not shown in the figures.

The mutual engagement of the latching elements 53 with the latching structures 33 in the second extended position of the cover structure 5, as shown in FIG. 14, effectively prevents another retraction of the cover structure 5 relative to the housing 3. The retraction is particularly prevented due to a distally oriented stop surface of the latching element 53 that abuts a proximally oriented stop surface of the latching element 33 in each case. Thus, the latching element 33 forms a retaining element for retaining the cover structure 5 from being retracted. Since in the present embodiment, the latching element 33 is formed by the housing 3, the housing 3 can be regarded as a retaining structure.

Access for the user to the site where the latching elements 33 and 53 are mutually engaged in the second extended position of the cover structure is prevented by the presence of the cylindrical wall of the housing 3. The cylindrical wall of the housing 3 form an envelope surface 32, which covers both latching element 33 and the latching element 53 in the radial direction outwards. Consequently, the user has no possibility to unlock the cover structure 5 with respect to the housing 3 and to move the cover structure 5 towards it retracted position.

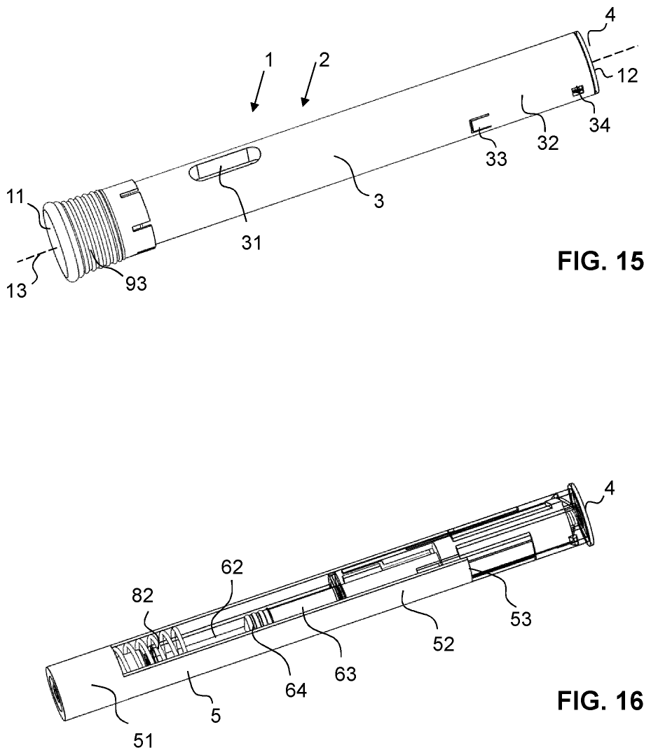

FIGS. 15 to 21 show a second example of a medicament delivery device 1 such as an autoinjector according to another inventive embodiment. In the following, the second example of FIGS. 15 to 21 is explained primarily in terms of its differences from the first example of FIGS. 1 to 14.

As it is shown in FIG. 15, a front cap 93 can be provided, which is attached to the proximal end of the housing 3, in order to secure the medicament delivery device 1 prior to its application. The front cap 93 can also serve to guarantee integrity and sterility of the medicament delivery device 1 before use. For the injection, the front cap 93 is removed from the housing 3 by the user.

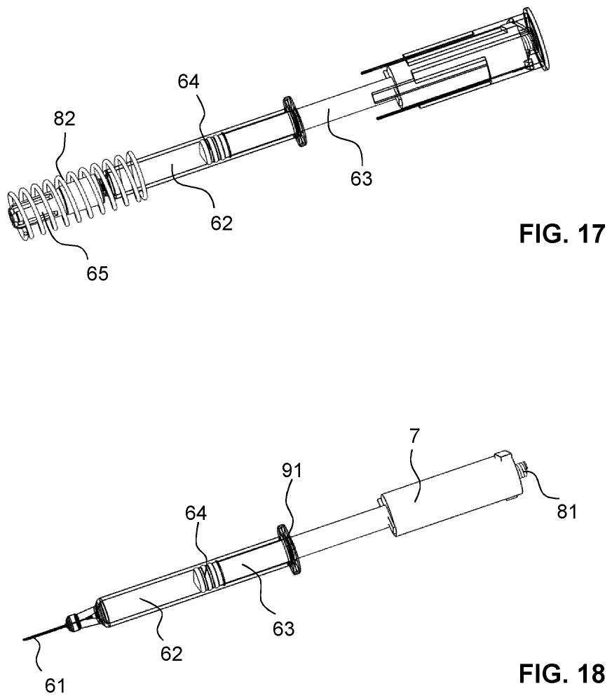

In FIGS. 16 and 17, a cover spring 82 is shown, which serves to bias the cover structure 5 in the proximal direction.

The embodiment of FIGS. 15 to 21 particularly differs from the one of FIGS. 1 to 14 by the design of the latching elements 33 and 53. As can be seen in FIG. 15, the latching elements 33 of the housing 3 are formed in each case (i.e. on the two diametrically opposed sides of the housing 3) by a proximally extending arm which is formed within a rectangular cut-out in the side wall of the housing 3. In an unstressed state, i.e. if the housing 3 is for example separate from all other components of the medicament delivery device 1, the arms formed by the latching elements 33 extend slightly radially inwards, as it is shown FIG. 21. It is noted that the housing 3 thus also forms a retaining structure here.

The latching elements 53 of the cover structure 5 are formed by the distal rims of the distally extending guide arms 52 of the cover structure 5.

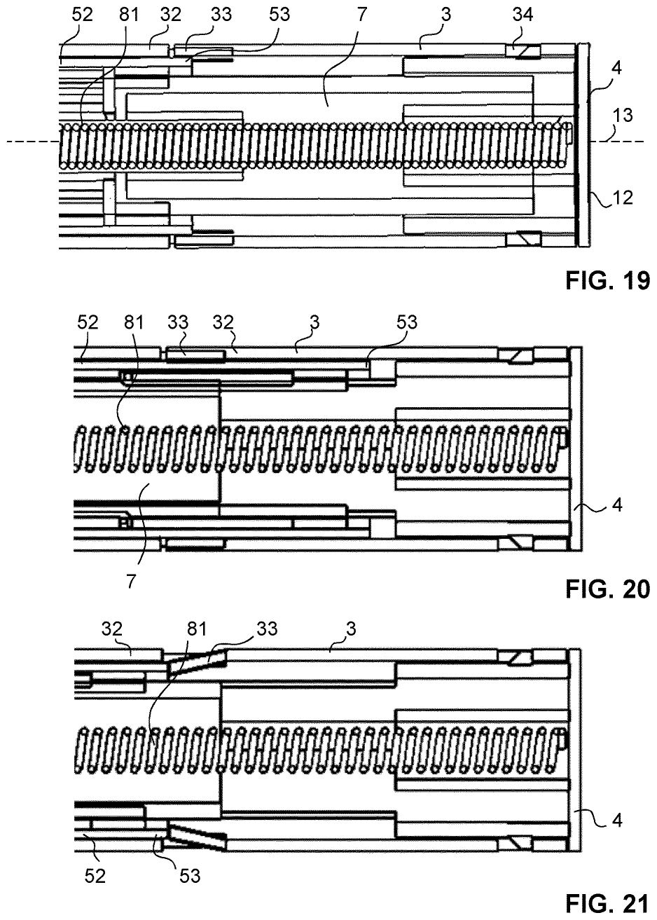

In the first extended position, as it is shown in FIG. 19, the distal rims of the guide arms 52, i.e. the latching structures 53, are positioned distally with respect to the latching elements 33 of the housing 3. The latching elements 33 rest against the outer surfaces of the guide arms 52, which push the latching elements 33 radially outwards. As a result, the cover structure 5 can be moved in the distal direction towards its retracted position shown in FIG. 20.

FIG. 20 shows the situation immediately after injection, i.e. after release of the injection spring 81, with the cover structure 5 in its retracted position. Due to the bias of cover spring 82, the cover structure 5 does not remain in the retracted position as shown in FIG. 20, but is moved proximally towards the second extended position, which is shown in FIG. 21.

The proximal movement of the cover structure 5 relative to the housing 3 is eventually stopped by a stop surface of e.g. the housing 3 not shown in the figures. In this position, as shown in FIG. 21, the distal rims of the guide arms 52, i.e. the latching elements 53, are positioned proximally of the latching elements 33. The latching elements 33, which are snap-in elements, do not rest against the outer surfaces of the guide arms 52 anymore and are allowed to flex radially inward, in order to adopt an unstressed state. As a result, when moving the cover structure 5 back towards its retracted position, the latching structures 53 distally abut against latching elements 33, by which a further distal movement of the cover structure 5 towards the retracted position is effectively prevented.

In the regions of the latching elements 33, the cylindrical wall of the housing 3 forms an envelope surface 32 that laterally surrounds the latching elements 33 in each case. Since the latching elements 33 of the housing 3 are each arranged within a cut-out of the cylindrical wall of the housing 3 and are, thus, laterally surrounded by an envelope surface 32 in each case, access for a user to the site of engagement of the two latching elements 33, 53 is made considerably more difficult. In particular when using his own hands, the user is not able to disengage the latching elements 33 and 53, since he does not have lateral access to the latching elements 33 of the housing 3 and, therefore, is not able to grasp and radially pull out the latching elements 33, in order to remove them from the latching elements 53.

The inventive concept has mainly been described above with reference to a few examples. However, as is readily appreciated by a person skilled in the art, other embodiments than the ones disclosed above are equally possible within the scope of the inventive concept, as defined by the appended claims. It is particularly to be noted that the combinations of elements, components and parts as shown in the figures are merely to be understood as non-limiting examples. The individual elements, components and parts of the various embodiments as described and/or shown in the figures can basically be interchanged with each other as desired and supplemented, for example, with further elements. Various modifications to the embodiments described are possible and will occur to those skilled in the art without departing from the invention which is defined by the following claims.

1-13. (canceled)

14. A subassembly of a medicament delivery device for delivering a medicament from a medicament container to a human or animal patient, the medicament delivery device extending axially and having, at a proximal end, a medicament delivery element through which the medicament can be delivered to the patient, wherein the subassembly comprises:
a cover structure configured to surround the medicament delivery element and having a first latching element, the cover structure being movable along the axial direction from a proximal first extended position to a distal retracted position and to a proximal second extended position, in order to cover the medicament delivery element in the first extended position before medicament delivery and in the second extended position after medicament delivery and to expose the medicament delivery element in the retracted position during medicament delivery; and
a retaining structure having an envelope surface arranged radially outside of the cover structure and having a second latching element,
wherein in the first extended position, the first latching element is out of engagement and positioned distally with respect to the second latching element,
wherein in the second extended position, the first latching element is in mutual engagement with respect to the second latching element, such that the cover structure is prevented from moving towards the retracted position, and
wherein in the second extended position, the envelope surface covers or surrounds the first latching element and/or the second latching element, in order to prevent disengagement of the first latching element and the second latching element by a user.

15. The subassembly of claim 14, wherein the first latching element (53) or the second latching element (33) is a snap-in element.

16. The subassembly of claim 15, wherein the first latching element is the snap-in element and the second latching element is formed by a depression on an inner face of the envelope surface of the retaining structure.

17. The subassembly of claim 16, wherein snap-in element is formed by a radially outwardly oriented flexible hook.

18. The subassembly of claim 16, wherein an axial guide groove is provided on the inner face of the envelope surface, in order to guide the snap-in element in the movement of the cover structure from the first extended position to the retracted position.

19. The subassembly of claim 18, wherein the snap-in element has a chamfered surface on its side facing in the proximal direction and/or the axial guide groove is axially terminated in the proximal direction by a chamfered surface.

20. The subassembly of claim 15, wherein the second latching element is the snap-in element, which is formed by a flexible arm.

21. The subassembly of claim 20, wherein the snap-in element is formed within an aperture of the envelope surface.

22. The subassembly of claim 20, wherein in an unstressed state, the snap-in element extends radially inwards from the envelope surface.

23. The subassembly of claim 14, wherein the retaining structure is formed by an outer housing of the medicament delivery device.

24. The subassembly of claim 14, wherein the cover structure has two or more first latching elements and the retaining structure has two or more second latching elements, such that two or more pairs of a first latching element and a second latching element are provided, which are regularly distributed along a circumferential direction of the subassembly.

25. The subassembly of claim 14, wherein the cover structure and/or the retaining structure are each made as a whole in one piece and preferably by means of injection moulding.

26. A medicament delivery device comprising the subassembly of claim 14.
